(19) United States
(12) Patent Application Publication
(10) Pub. No.: US 2016/0345519 A1
(43) Pub. Date: Dec. 1, 2016

(54) BIDENS PLANT HAVING UNIQUE COLORATION

(71) Applicant: Koichiro Nishikawa, Okayama-ken (JP)

(72) Inventor: Koichiro Nishikawa, Okayama-ken (JP)

(21) Appl. No.: 15/218,355

(22) Filed: Jul. 25, 2016

Related U.S. Application Data
(63) Continuation-in-part of application No. 13/839,509, filed on Mar. 15, 2013, now abandoned.
Application No. 13/839,509 is a continuation-in-part of application No. 13/507,488, filed on Jul. 3, 2012, now Pat. No. PP25087.
Application No. 13/507,488 is a continuation-in-part of application No. 13/507,783, filed on Jul. 27, 2012, now Pat. No. PP24866.
Application No. 13/507,783 is a continuation-in-part of application No. 13/507,791, filed on Jul. 27, 2012, now Pat. No. PP24867.

(30) Foreign Application Priority Data
Mar. 27, 2012 (QZ) 2012/0695
May 16, 2012 (QZ) 2012/1079
May 16, 2012 (QZ) 2012/1080

(51) Int. Cl. A01H 5/02; A01H 1/02

(52) U.S. Cl. CPC A01H 5/025; A01H 1/02

(57) ABSTRACT

A plant material and methodology for producing Bidens with any primary color or intermediate color, in unique petal coloration patterns are provided herein.

### TECHNICAL FIELD

The present disclosure relates generally to the field of ornamental Bidens plants (Bidens triplinervia) and provides methodology and materials for creating new, distinct, and stable Bidens cultivars with unique ray floret coloration and patterns. Such Bidens plants have not been observed in any wild species of Bidens, but was discovered and developed through Applicant's breeding process.

### INTRODUCTION

Bidens is a genus of flowering plants in the family Asteraceae, and there are about 200 Bidens species. Common names beggarticks, black jack, burr marigolds, cobbler's pegs, and Spanish needles, all refer to achene burrs on the seeds, most of which are barbed. The generic name “Bidens” refers to the same fact; it means “two-tooth,” from Latin bis“two”+dens “tooth”. The plants are Zoochorous; their seeds will stick to clothing, fur or feathers, which allows direct transport to a new habitat. This has enabled Bidens to colonize a wide range, including many oceanic islands. Consequently, some Biden species occur only in a very restricted range and several face extinction, notably in remote islands. Due to the absence of native mammals on these islands, some of the oceanic island taxa have reduced burrs, evolving features that may facilitate wind dispersal. Bidens are characterized by white or yellow mono-color petals.

### SUMMARY

Provided herein are plant material and methodology for producing Bidens with any primary color or intermediate color, in unique petal coloration patterns.

In one aspect, provided is a Bidens plant comprising: ray florets and disc florets, each ray floret having an upper surface divided into a plurality of regions; wherein the Bidens plant belongs to Bidens triplinervia or any progeny of Bidens triplinervia; and wherein a color of each region is controlled based on a region-by-region approach that crosses a Bidens plant comprising ray floret coloration and patterns according to the plurality of regions with a Bidens plant comprising or not comprising ray floret coloration and patterns according to the plurality of regions.

In another aspect, provided is said Bidens plant, wherein the plurality of regions includes first regions, a second region, a third region, and fourth regions, the second region being interposed between the first regions, the third region being interposed between the fourth regions, and the third region and the fourth regions being adjacent to the first regions and the second region along an axis.

In another aspect, provided is the Bidens plant, wherein the color of each region is selected from a group consisting of red, yellow, orange, white, and lemon, and the color of at least one of the plurality of regions is red or orange.

In another aspect, provided is the Bidens plant, wherein the color of each region is selected from a group consisting of red, yellow, orange, white, lemon, black, and green, and the color of at least one of the plurality of regions is red, orange, or black.

In another aspect, provided is the Bidens plant, wherein at least one of the plurality of regions is brushed or brushed with another color.

In another aspect, provided is the Bidens plant, wherein the color of the first regions and the second region is the same, the color of the third region and the fourth regions is the same, and the third region and the fourth regions are brushed by another color.

In another aspect, provided is the Bidens plant, wherein the color of the third region and the fourth regions is the same, the color of the first regions and the second region is the same, the color of the first regions and the second region is different from the color of the third region and the fourth regions, and the second region is brushed by the color of the third region and the fourth regions.

In another aspect, provided is the Bidens plant, wherein the color of the first regions and the fourth regions is the same and the color of the second region and the third region is different from the color of the first regions and the fourth regions.

In another aspect, provided is the Bidens plant, the color of at least one of the plurality of regions is gradually changing from beginning to bloom towards ending to bloom.

In another aspect, provided is the Bidens plant, wherein the plurality of regions includes first regions, a second region, a third region, fourth regions, a fifth region, and sixth regions, the second region being interposed between the first regions, the third region being interposed between the fourth regions, the fifth region being interposed between the sixth regions, and the first and the second regions being interposed between the third and the fourth regions and the fifth and the sixth regions.

In another aspect, provided is the Bidens plant, wherein the color of each region is selected from a group consisting of red, yellow, orange, white, and lemon, and the color of at least one of the plurality of regions is red or orange.

In another aspect, provided is the Bidens plant, wherein the color of each region is selected from a group consisting of red, yellow, orange, white, lemon, black, and green, and the color of at least one of the plurality of regions is red, orange, or black.

In another aspect, provided is the Bidens plant, wherein at least one of the plurality of regions is brushed or brushed with another color.

In another aspect, provided is the Bidens plant, wherein the color of the first regions, the second region, and the fourth regions is yellow, the color of the third region is red, and the color of the fifth region and sixth regions is lemon.

In another aspect, provided is the Bidens plant, wherein the color of the first regions and the second region is yellow, the color of the third region and the fourth regions is red, the color of the fifth region and the sixth regions is lemon.

In another aspect, provided is the Bidens plant, wherein the color of the first regions, the second region, the fifth region, and the sixth regions is red and the color of the third region and the fourth regions is yellow.

In another aspect, provided is the Bidens plant, wherein the color of the first regions, the second region, the third region, and the fourth regions is red and the color of the fifth region and the sixth regions is lemon or lemon with red brushing.

In another aspect, provided is the Bidens plant, wherein the Bidens plant coloration and color patterns are stably and predictably introgressed into diverse Bidens genetic background.

In another aspect, there is provided methodology for producing the Bidens plant, comprising: (a) crossing a first Bidens plant comprising characteristics of the Bidens, with a second Bidens plant that comprises or does not comprise the characteristics of the Bidens, and (b) selecting progeny having the characteristics of the Bidens.

In another aspect, provided is a method for asexually reproducing the Bidens plant, comprising: (a) obtaining a tissue cutting from said plant, (b) culturing said tissue cutting under conditions sufficient to produce a plantlet with roots and shoots; and (c) growing said plantlet to produce a plant.

### BRIEF DESCRIPTION OF THE FIGURES

This patent or application file contains at least one drawing executed in color. Copies of this patent or patent application publication with color drawing(s) will be provided by the Office upon request and payment of necessary fee.

FIG. 1 shows Bidens plant ‘Florbikanoyel’ with orange and yellow brush coloration.

FIG. 2 shows Bidens plant ‘Florbikanre’ with light red-colored ray florets.

FIG. 3 shows Bidens plant ‘Florbikanyo’ with yellow orange and dark orange bi-colored ray florets.

FIG. 4 shows an illustrative Bidens coloration pattern, pattern A. For example, region 2 could be colored red, with regions 1, 3, and 4 maintained as yellow. Likewise, regions 1 and 4 could be colored red, with regions 2 and 3 maintained as yellow.

FIG. 5 shows an illustrative Bidens coloration pattern, pattern B. For example, the petal can be bi-color.

FIG. 6 shows an illustrative Bidens coloration pattern, pattern C. Here, the pattern provides a sectional mix of pattern B disclosed in FIG. 5.

FIG. 7 shows an illustrative Bidens coloration pattern, pattern D Here, the pattern provides a variation of pattern B disclosed in FIG. 5.

FIG. 8 shows a Bidens double flower pattern. In double flower Bidens, the internal petal set may have one color, and the external set may have a different color. For example, and non-limiting, the internal petal set may have a mono-color, whereas the external petal set may have multi-colors.

FIG. 9 shows an illustrative Bidens coloration pattern according to Example 6.

FIG. 10 shows Bidens plants with unique ray floret coloration and patterns according to Example 6.

FIG. 11 shows Bidens plants with unique ray floret coloration and patterns according to Example 6.

FIG. 12 shows Bidens plants with unique ray floret coloration and patterns according to Example 6.

FIG. 13 shows Bidens plants with unique ray floret coloration and patterns according to Example 6. And the figures depict one or more implementations in accordance with the present teaching, by way of example only, not by way of limitations.

### DETAILED DESCRIPTION

All Bidens ferulifolia or any Bidens hybrid in the market has white or yellow mono-color petals. The present inventor discovered and developed the first Bidens varieties with unique coloration, including, for example, red, orange and/or multi-color petals. From these introductions and using breeding methodology that can control flower petal color on a section-by-section approach, the present inventor can introduce any primary color (red, black, yellow, green, white) or any intermediate color (orange, lemon, etc.) to create many petal colors and petal designs. Thus, and as described below, the instant application provides methodology and materials for creating new, distinct, and stable Bidens cultivars with unique ray floret coloration and patterns.

As used herein, multi-color refers to the introduction of one or more colors on Bidens petal. Unlike conventional Bidens that are have only white or yellow mono-color petals, Applicant has developed breeding material and methodology for introducing one or more colors into ray florets. In no way limiting, Applicant has developed new colors aside from traditional white and yellow including exemplary primary colors red, green, and black, as well as exemplary intermediate colors produced by mixing at least two primary colors. Illustrative intermediate colors include orange and lemon, for example. Additionally, and as used herein, a Bidens plant may have petals with bi-color coloration, such as instant Bidens plant ‘Florbikanyo’ with yellow orange and dark orange bi-colored ray florets.

“Plant” includes plant cells, plant protoplasts, plant cells of tissue culture from which Bidens plants can be regenerated from plant calli, plant clumps and plant cells that are intact in plants or parts of plants such as pollen, flowers, pistils, anthers, seeds, leaves, stems, and the like.

A. Identification of Bidens plant ‘Florbikanre’

The present disclosure embraces a new and distinct cultivar of Bidens plant, botanically known as Bidens triplinervia, and hereinafter named ‘Florbikanre.’

Florbikanre′ originated from a self-pollination made by the Inventor in Okayamaken, Japan in January, 2010 of Bidens triplinervia ‘KR-SAIKA001’, not patented. The new Bidens plant was discovered and selected by the Inventor as a single flowering plant from within the progeny of the stated self-pollination in a controlled greenhouse environment in Okayama-ken, Japan in May, 2010.

Asexual reproduction of ‘Florbikanre’ by vegetative cuttings in a controlled environment in Okayama-ken, Japan since June, 2010 has shown that the unique features of ‘Florbikanre’ are stable and reproduced true to type in successive generations.

‘Florbikanre’ have not been observed under all possible environmental conditions and cultural practices. The phenotype may vary somewhat with variations in environmental conditions such as temperature and light intensity, without, however, any variance in genotype. The following traits have been repeatedly observed and are determined to be the unique characteristics of ‘Florbikanre’. These characteristics in combination distinguish ‘Florbikanre’ as a new and distinct Bidens plant:

1. Large, upright, outwardly spreading to somewhat trailing and mounding plant habit.
2. Vigorous growth habit.
3. Freely branching habit.
4. Freely flowering habit.
5. Long flowering period.
6. Inflorescences with light red-colored ray florets and yellow-colored disc florets.
7. Strong peduncles that hold the inflorescences above and beyond the foliar plane.

Plants of ‘Florbikanre’ differ primarily from plants of the parent, ‘KR-SAIKA001’ in ray floret color, as inflorescences of the new ‘Florbikanre’ Bidens have light red-colored ray florets whereas inflorescences of ‘KRSAIKA001’ have subdued red-colored ray florets.

A complete botanical description of ‘Florbikanre’ can be found in U.S. Plant patent application Ser. No. 13/507,791R. Representative ‘Florbikanre’ seeds are deposited with the American Type Culture Collection under deposit designation XXX.

B. Identification of Bidens plant ‘Florbikanoyel’

The present disclosure embraces a new and distinct cultivar of Bidens plant, botanically known as Bidens triplinervia, and hereinafter named ‘Florbikanoyel’ ‘Florbikanoyel’ originated from a self-pollination made by the Inventor in Okayamaken, Japan in January, 2010 of Bidens triplinervia ‘KR-SAIKA001’, not patented. Florbikanoyel was discovered and selected by the Inventor as a single flowering plant from within the progeny of the stated self-pollination in a controlled greenhouse environment in Okayama-ken, Japan in May, 2010.

Asexual reproduction of ‘Florbikanoyel’ by vegetative cuttings in a controlled environment in Okayama-ken, Japan since June, 2010 has shown that the unique features of ‘Florbikanoyel’ are stable and reproduced true to type in successive generations.

‘Florbikanoyel’ have not been observed under all possible environmental conditions and cultural practices. The phenotype may vary somewhat with variations in environmental conditions such as temperature and light intensity, without, however, any variance in genotype. The following traits have been repeatedly observed and are determined to be the unique characteristics of ‘Florbikanoyel’. These characteristics in combination distinguish ‘Florbikanoyel’ as a new and distinct Bidens plant:

1. Large, upright, outwardly spreading to somewhat trailing and mounding plant habit.
2. Vigorous growth habit.
3. Freely branching habit.
4. Freely flowering habit.
5. Long flowering period.
6. Inflorescences with orange-colored ray florets with a “brush” of yellow towards the base and yellow-colored disc florets.
7. Strong peduncles that hold the inflorescences above and beyond the foliar plane.

Plants of ‘Florbikanoyel’ differ primarily from plants of the parent, ‘KR-SAIKA001’ in ray floret color, as inflorescences of the new ‘Florbikanoyel’ Bidens have orange-colored ray florets with a “brush” of yellow towards the base of the floret, whereas inflorescences of ‘KRSAIKA001’ have yellow and subdued orange bi-colored ray florets.

A complete botanical description of ‘Florbikanoyel’ can be found in U.S. Plant patent application Ser. No. 13/507,783R. Representative ‘Florbikanoyel’ seeds are deposited with the American Type Culture Collection under deposit designation XXX.

C. Identification of Bidens plant ‘Florbikano’

The present disclosure embraces a new and distinct cultivar of Bidens plant, botanically known as Bidens triplinervia, and hereinafter named ‘Florbikano’

‘Florbikano’ originated from a self-pollination made by the Inventor in Okayama-ken, Japan in January, 2010 of Bidens triplinervia ‘KR-SAIKA001’, not patented. Florbikano was discovered and selected by the Inventor as a single flowering plant from within the progeny of the stated self-pollination in a controlled greenhouse environment in Okayama-ken, Japan in May, 2010.

Asexual reproduction of ‘Florbikano’ by vegetative cuttings in a controlled environment in Okayama-ken, Japan since June, 2010 has shown that the unique features of ‘Florbikano’ are stable and reproduced true to type in successive generations.

‘Florbikano’ have not been observed under all possible environmental conditions and cultural practices. The phenotype may vary somewhat with variations in environmental conditions such as temperature and light intensity, without, however, any variance in genotype. The following traits have been repeatedly observed and are determined to be the unique characteristics of ‘Florbikano’. These characteristics in combination distinguish ‘Florbikano’ as a new and distinct Bidens plant:

1. Large, upright, outwardly spreading to somewhat trailing and mounding plant habit.
2. Vigorous growth habit.
3. Freely branching habit.
4. Freely flowering habit.
5. Long flowering period.
6. Inflorescences with yellow orange and dark bi-colored ray florets and bright yellow-colored disc florets.
7. Strong peduncles that hold the inflorescences above and beyond the foliar plane.

Plants of ‘Florbikano’ differ primarily from plants of the parent, ‘KR-SAIKA001’ in ray floret color, as inflorescences of the new ‘Florbikano’ Bidens have orange and dark bi-colored ray florets, whereas inflorescences of ‘KRSAIKA001’ have yellow and subdued orange bi-colored ray florets.

A complete botanical description of ‘Florbikano’ can be found in U.S. Plant patent application Ser. No. 13/507,488R. Representative ‘Florbikano’ seeds are deposited with the American Type Culture Collection under deposit designation.

### D. Stable and Predictable Introgression of Bidens Color

The instant Bidens coloration and pattern can be stably and predictably introgressed into diverse Bidens genetic backgrounds. The instant Bidens can be used as a male or female parent in crosses for introducing the trait into new Bidens plants, thereby creating diverse Bidens genetic backgrounds.

Additionally, and as known in the art, Bidens plants can be reproduced asexually by vegetative propagation or other clonal method known in the art. For example, and in no way limiting, a Bidens plant having at least one inflorescence with red ray florets, can be reproduced by (a) obtaining a tissue cutting from said plant, (b) culturing said tissue cutting under conditions sufficient to produce a plantlet with roots and shoots; and (c) growing said plantlet to produce a plant.

### E. Developing New Coloration in Bidens Plants

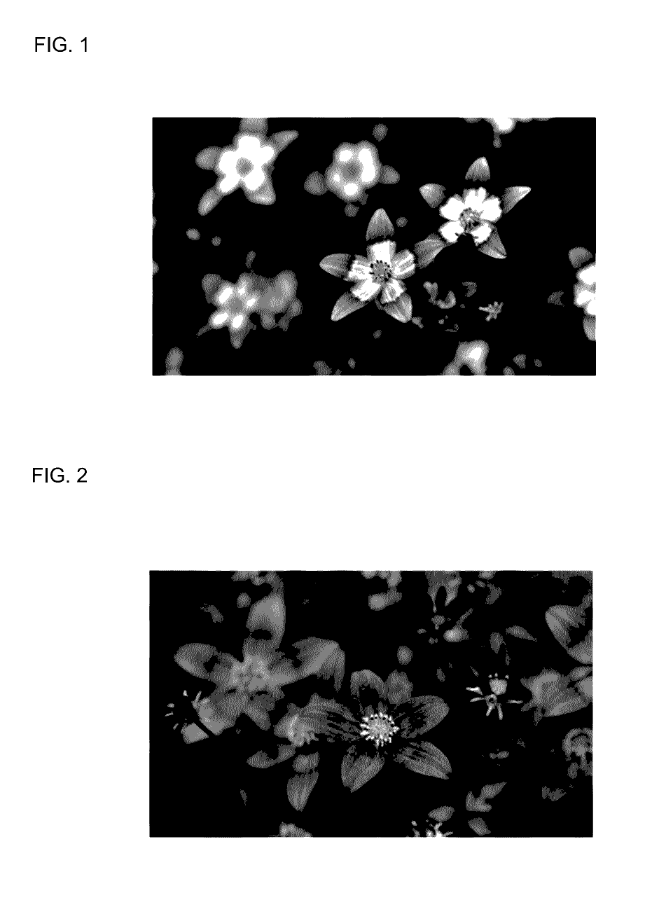

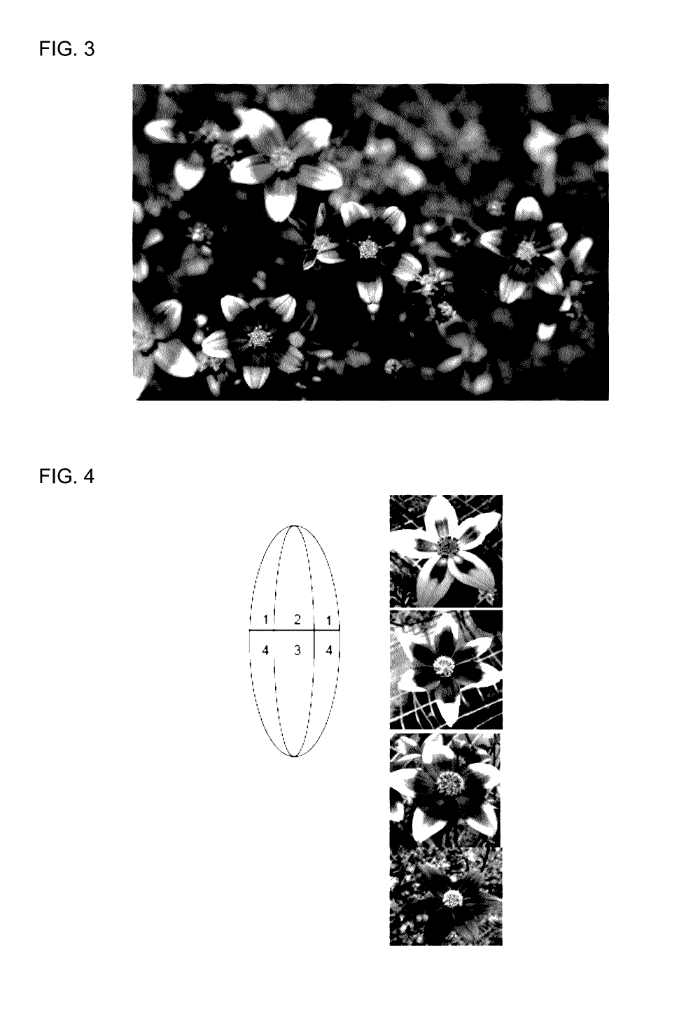

Using the instant Bidens plant material and breeding methodology that can control flower petal color on a section-by-section approach, the present inventor can introduce any primary color (red, black, green, white, yellow) or any intermediate color (orange, lemon, etc.) to create many petal colors and petal designs. Exemplary coloration and patterns are illustrated in FIGS. 1-8.

The examples presented below disclose details concerning exemplary Bidens plants with novel petal colors, as well as methodology for producing Bidens plants with unique petal color patterns. The examples are illustrative and nonlimiting.

Further, following points shall be taken into consideration. Bidens pilosa L. (for example, the variety denomination is Pirate's Princess), Bidens laevis(for example, the variety denomination is Winter Cosmos), and Bidens aurea belong to Bidens, but these Bidens are different from a group of Bidens triplinervia and Bidens ferulifolia according to plant classification. It is a well known fact that Bidens plant is usually divided into two types, that is, the group of Bidens ferulifolia and the group of Bidens laevis. Botanically, the group of Bidens laevis is separated from the group of Bidens ferulifolia or Bidens triplinervia, because the group of Bidens ferulifolia has different characteristics from the group of Bidens laevis.

More specifically, (1) Bidens pilosa L., Bidens laevis, and Bidens aurea cannot cross with Bidens triplinervia or Bidens ferulifolia to create any hybrid Bidens. (2) The group of Bidens laevis comprises ray florets whose extention are obovate in shape and their outer edges are shallowly cleft. On the other hand, the group of Bidens triplinervia and Bidens ferulifolia does not have such shape of ray florets at its edge. (3) The height of Bidens laevis sometimes reaches over 1 m, while the height of Bidens triplinervia group reaches about 40 cm and trail along the ground. In other words, Bidens triplinervia group is low-growing, while Bidens laevis group is not low-growing. (4) Bidens pilosa L., Bidens laevis, and Bidens aurea are weak to cold, while Bidens triplinervia and Bidens ferulifolia are strong to cold. (5) A plant life of Bidens pilosa L., Bidens laevis, and Bidens aurea is short, while a plant life of Bidens triplinervia and Bidens ferulifolia is comparatively long.

Moreover, although Bidens pilosa L., Bidens laevis, and Bidens aurea belong to Bidens plant, they are very similar to cosmos. Therefore, it is a well-known fact that Bidens laevis with red-purple colored ray florets has already existed.

### Example 1

### Botanical Description of ‘Florbikano’

Plants of the new Bidens have not been observed under all possible environmental conditions and cultural practices. The phenotype may vary somewhat with variations in environmental conditions such as temperature and light intensity, without, however, any variance in genotype. The following traits have been repeatedly observed and are determined to be the unique characteristics of ‘Florbikano’. These characteristics in combination distinguish ‘Florbikano’ as a new and distinct Bidens plant:

1. Upright, outwardly spreading to trailing and mounding plant habit.
2. Vigorous growth habit.
3. Freely branching habit.
4. Freely 5 flowering habit.
5. Long flowering period.
6. Inflorescences with yellow orange and dark orange bicolored ray florets and bright yellow-colored disc florets.
7. Strong peduncles that hold the inflorescences above and 10 beyond the foliar plane.

Plants of the new Bidens differ primarily from plants of the parent, ‘K-SAIKA001’ in ray floret color as inflorescences of the new Bi dens have yellow orange and dark orange bi-colored ray florets whereas inflorescences of ‘K-SAIKA001’ have yellow and subdued orange bi-colored ray florets.

Plants of the new Bidens can be compared to plants of Bidens triplinervia ‘Yellow Charm’, not patented. In side-by-side comparisons conducted in Okayama-ken, Japan, plants of the new Bidens differed primarily from plants of ‘Yellow Charm’ in the following characteristics:

1. Plants of the new Bidens were larger than plants of Yellow Charm′.
2. Plants of the new Bidens were more outwardly spreading and trailing than and not as upright as plants of ‘Yellow Charm’.
3. Plants of the new Bidens had narrower and darker green colored leaves than plants of ‘Yellow Charm’.
4. Plants of the new Bidens and ‘Yellow Charm’ differed in ray floret color as plants of ‘Yellow Charm’ had solid yellow colored ray florets.
5. Plants of the new Bidens were more high temperature tolerant than plants of ‘Yellow Charm’.

### DETAILED BOTANICAL DESCRIPTION

The following observations and measurements describe plants grown in 30-cm containers during the late spring in a polyethylene-covered greenhouse in Okayama ken, Japan and under cultural practices which approximate those generally used in commercial Bidens production. During the production of the plants, day temperatures ranged from 20° C. to 30° C. and night temperatures ranged from 5° C. to 10° C. Plants were one year old when the photographs and description were taken. In the following description, color references are made to The Royal Horticultural Society Colour Chart, 2007 Edition, except where general terms of ordinary dictionary significance are used.

### Botanical Classification:

Bidens triplinervia ‘Florbikano’.

### Parentage:

Self-pollination of Bidens triplinervia ‘K-SAIKA001’, not patented.

### Propagation:

Type: By vegetative cuttings.

Time to initiate roots, summer: About two weeks at about 20° C. to 30° C.
Time to initiate roots, winter: About three weeks at about 10° C. to 15° C.
Time to produce a rooted young plant, summer: About three weeks at about 20° C. to 30° C.
Time to produce a rooted young plant, winter: About four weeks at about 10° C. to 15° C.
Root description: Fine, fibrous; 5 white in color.
Rooting habit: Freely branching; dense.

### Plant Description:

Plant and growth habit: Upright, outwardly spreading to trailing and mounding plant habit; vigorous growth habit.

Branching habit: Freely branching habit with lateral branches potentially forming at every node.
Plant height: About 30 cm.
Plant diameter or spread: About 100 cm.

### Lateral Branches:

### 15 Length: About 10 cm.

### Diameter: About 1 mm.

Internode length: About 10 cm.

Strength: Strong, flexible.
Texture: Slightly pubescent.

### Color: Close to 144A.

### Foliage Description:

Arrangement: 5 Opposite, simple.

### Length: About 3.5 cm.

### Width: About 2.5 cm.

Shape: Roughly deltoid; pinnatisect.

### Apex: Acute.

### Base: Acute.

Margin: Deeply incised; laciniate.

Texture, upper and lower surfaces: Smooth, glabrous.
Venation pattern: Pinnate.

### Color:

Developing leaves, upper surface: Close to 137A.

Developing leaves, lower surface: Close to 138A.
Fully expanded leaves, upper surface: Close to NI37A;
venation, close to 137A.
Fully expanded leaves, lower surface: Close to 137C;
venation, close to N137C.

### Petioles:

### Length: About 9 mm.

### Diameter: About 4 mm.

Texture, upper surface: Sparsely pubescent.

Texture, lower surface: Smooth, glabrous.
10 Color, upper and lower surfaces: Close to 146C.

### Inflorescence Description:

Appearance: Single (daisy) inflorescence form with ray and disc florets; inflorescences positioned above and beyond the foliar plane on strong peduncles; inflorescences face upright to outwardly.

Flowering habit: Freely flowering habit with numerous inflorescences developing per plant.
Fragrance: None detected.
Natural flowering season: Long flowering period, plants flower continuously from spring until the autumn in Japan.
Inflorescence longevity: Inflorescences last about one Week
on the plant; inflorescences persistent.

### Inflorescence Buds:

### Height: About 3 mm.

### Diameter: About 3 mm.

### Shape: Ovoid.

### 10 Color: Close to 151A.

### Inflorescence Size:

### Diameter: About 3.5 cm.

Depth (height): About 1 cm.

Disc diameter: About 4 mm.
Receptacle diameter: About 1.2 cm.
Receptacle height: About 4 mm.
Receptacle color: Close to 144B.

### Ray Florets:

### Length: About 1.7 cm.

### Width: About 1.1 cm.

### Shape: Ovate.

### Apex: Emarginate.

### Base: Obtuse.

### Margin: Entire.

Texture, upper and lower surfaces: Smooth, glabrous.

Number of ray florets per inflorescence: About five arranged in a single whorl.

### Color:

When opening, upper surface: Towards the apex, close to N25C; towards the base, close to N34A.

When opening, lower surface: Close to 163A.
Fully opened, upper surface: Towards the apex, close to 23A; towards the base, close to 34A; with development, color towards the apex becomes closer to 17C and towards the base, close to 168A.
Fully opened, lower surface: Close to 163A; with development, color becomes closer to 162A.

### Disc Florets:

Shape: Tubular; apex dentate.

### Length: About 5 mm.

### Diameter: About 1 mm.

Number of disc florets per inflorescence: About 42.

Color, when Opening:

### Apex: Close to 12B.

### Mid-section: Close to 161A.

### Base: Close to 145C.

### Color, Fully Opened:

### Apex: Close to 14A.

### Mid-section: Close to 151A.

### Base: Close to 145B.

### Phyllaries:

Quantity per inflorescence: About six in a single whorl.

### Length: About 3 mm.

### Width: About 0.6 mm.

### Shape: Lanceolate.

### Apex: Acute.

### Base: Truncate.

### Margin: Entire.

Color, upper and lower surfaces: Close to 137B.

Texture, upper and lower surfaces: Smooth, glabrous.

### Peduncles:

Length, terminal peduncle: About 7 cm.

Length, fourth peduncle: About 10.2 cm.

### Diameter: About 1 mm.

Strength: Strong; flexible.

Aspect: Upright to outwardly holding inflorescences above and beyond the foliar plane.
Texture: Smooth, glabrous.

### Color: Close to 146A.

### Reproductive Organs:

Androecium: Present on disc florets only.

Quantity per 5 disc floret: One.
Filament length: About 1.5 mm.
Filament color: Close to 145D.
Anther shape: Lanceolate.
Anther length: About 1.2 mm.
Anther color: Close to 200A.
Pollen amount: Scarce.
Pollen color: Close to 21A.
Gynoecium: Present on ray and disc florets.
Pistil length: About 7 mm.
Style length: About 5 mm.
Style color: Close to 145D.
Stigma shape: Bi-parted.
Stigma color: Close to 15A.
Ovary color: Close to 145D.
Seeds and fruits: Seed and fruit development have not been observed on plants of the new Bidens.

### Disease & Pest Resistance:

Plants of the new Bidens have not been shown to be resistant to pathogens and pests common to Bidens plants.

### Garden Performance:

Plants of the new Bidens have exhibited good tolerance to rain and wind and have been observed to tolerate temperatures from about 10 C. to about 47° C.

### Example 2

### Botanical Description of ‘Florbikanoyel’

Plants of the new Bidens have not been observed under all possible environmental conditions and cultural practices. The phenotype may vary somewhat with variations in environmental conditions such as temperature and light intensity, without, however, any variance in genotype.

The following traits have been repeatedly observed and are determined to be the unique characteristics of ‘Florbikanoyel’. These characteristics in combination distinguish ‘Florbikanoyel’ as a new and distinct Bidens plant:

1. Large, upright, outwardly spreading and mounding plant habit.
2. Vigorous growth habit.
3. Freely branching habit.
4. Freely 5 flowering habit.
5. Long flowering period.
6. Inflorescences with orange-colored ray florets with a “brush” of yellow towards the base and yellow-colored disc florets.
7. Strong peduncles that hold the inflorescences above and beyond the foliar plane.

Plants of the new Bidens differ primarily from plants of the parent, ‘K-SAIKA001’ in ray floret color as inflorescences of the new Bidens have orange-colored ray florets with a “brush” of yellow towards the base of the floret whereas inflorescences of ‘K-SAIKA001’ have yellow and subdued orange bi-colored ray florets.

Plants of the new Bidens can be compared to plants of Bidens triplinervia ‘Florbikano’, disclosed in U.S. Plant patent application Ser. No. 13/507,488. In side-by-side comparisons conducted in Okayama-ken, Japan, plants of the new Bidens differed from plants of ‘Florbikano’ in the following 5 characteristics:

1. Plants of the new Bidens were taller than plants of ‘Florbikano’.
2. Plants of the new Bidens had longer and thicker lateral branches than plants of ‘Florbikano’.
3. Plants of the new Bidens and ‘Florbikano’ differed in ray floret color as plants of ‘Florbikano’ had yellow orange and dark orange bi-colored ray florets.
4. Plants of the new Bidens had longer peduncles than plants of ‘Florbikano’.

### DETAILED BOTANICAL DESCRIPTION

The following observations and measurements describe plants grown in 40-cm containers during the late spring in a polyethylene-covered greenhouse in Okayama ken, Japan and under cultural practices which approximate those generally used in commercial Bidens production. During the production of the plants, day temperatures ranged from 20° C. to 30° C. and night temperatures ranged from 5° C. to 10° C. Plants were one year old when the photographs and description were taken. In the following description, color references are made to The Royal Horticultural Society Colour Chart, 2007 Edition, except where general terms of ordinary dictionary significance are used.

### Botanical Classification:

Bidens triplinervia ‘Florbikanoyel’.

### Parentage:

Self-pollination of Bidens triplinervia ‘K-SAIKA001’, not patented.

### Propagation:

Type: By vegetative cuttings.

Time to initiate roots, summer: About two weeks at temperatures about 20° C. to 30° C.
Time to initiate roots, winter: About three weeks at temperatures about 10° C. to 15° C.
Time to produce a rooted young plant, summer: About three weeks at temperatures about 20° C. to 30° C.
Time to produce a rooted young plant, winter: About four weeks at temperatures about 10° C. to 15° C.
Root description: Fine, fibrous; white in color.
Rooting habit: Freely branching; dense.

### Plant Description:

Plant and growth habit: Large, upright, outwardly spreading and mounding plant habit; vigorous growth habit.

Branching habit: Freely branching habit with lateral branches
potentially forming at every node.
Plant height: About 40 cm.
Plant diameter or spread: About 100 cm.

### Lateral Branches:

### Length: About 20 cm.

### Diameter: About 1.6 mm.

Internode length: About 11.5 cm.

Strength: Strong, flexible.
15 Texture: Slightly pubescent.
Color: Close to 144A tinted with close to N77A.

### Foliage Description:

Arrangement: Opposite, simple.

### Length: About 3.5 cm.

### Width: About 2.3 cm.

Shape: Roughly deltoid; 5 pinnatisect.

### Apex: Acute.

### Base: Acute.

Margin: Deeply incised; laciniate.

Texture, upper and lower surfaces: Smooth, glabrous.
Venation pattern: Pinnate.

### Color:

Developing leaves, lower surface: Close to 137D.

Developing leaves, upper surface: Close to N137D.
Fully expanded leaves, upper surface: Close to 137A, venation, close to 137A.
Fully expanded leaves, lower surface: Close to 137C, venation, close to 137B.

### Petioles:

### Length: About 1.4 cm.

### Diameter: About 0.9 mm.

Texture, upper surface: Sparsely pubescent.

Texture, lower surface: 5 Smooth, glabrous.
Color, upper surface: Close to 137B tinted with close to N77B.
Color, lower surface: Close to 137B.

### Inflorescence Description:

Appearance: Single (daisy) inflorescence form with ray and disc florets; inflorescences positioned above and beyond the foliar plane on strong peduncles; inflorescences face mostly upright to outwardly.

Flowering habit: Freely flowering habit with numerous inflorescences developing per plant.
Fragrance: None detected.
Natural flowering season: Long flowering period, plants flower continuously from spring until the autumn in Japan.
Inflorescence longevity: Inflorescences last about one week on the plant; inflorescences persistent.

### Inflorescence Buds:

### Height: About 4 mm.

### Diameter: About 3.5 mm.

### Shape: Ovoid.

### Color: Close to 151A and 152A.

### Inflorescence Size:

### Diameter: About 3.8 cm.

Depth (height): About 1 cm.

Disc diameter: About 3.5 mm.
Receptacle diameter: About 1.2 cm.
15 Receptacle height: About 3.5 mm.
Receptacle color: Close to 147A.

### Ray Florets:

### Length: About 1.9 cm.

### Width: About 1.3 cm.

### Shape: Ovate.

### 5 Apex: Acute.

### Base: Obtuse.

### Margin: Entire.

Texture, upper and loWer surfaces: Smooth, glabrous.

Number of ray florets per inflorescence: Five arranged in a single Whorl.

### Color:

When opening, upper surface: Towards the apex, close to 169A; towards the base, close to N163C.

When opening, lower surface: Towards the apex, 15 close to 169A; towards the base, close to 21A.
Fully opened, upper surface: Towards the apex, close to 169A; towards the base, close to 17A; with development, color towards the apex becomes closer to N163B and towards the base, close to 17B.
Fully opened, lower surface: Towards the apex, close to 169B; towards the base, close to 17B; with development, color towards the 5 apex becomes closer to N163B and towards the base, close to 17C.

### Disc Florets:

Shape: Tubular; apex dentate.

### Length: About 5 mm.

### Diameter: About 1 mm

Number of disc florets per inflorescence: About 39.

Color, when opening: Towards the apex, close to 1620; towards the base, close to 144C.
Color, fully opened: Towards the apex, close to 17B; towards the base, close to 144B.

### Phyllaries:

Quantity per inflorescence: Six in a single whorl.

### Length: About 4 mm.

### Width: About 0.8 mm.

### Shape: Lanceolate.

### Apex: Acute.

### Base: Truncate.

### Margin: Entire.

Texture, upper and lower surfaces: Smooth, glabrous.

Color, upper and lower surfaces: Close to 137A.

### Peduncles:

Length, terminal peduncle: About 12.5 cm.

### Diameter: About 1.3 mm.

Strength: Strong; flexible.

Aspect: Erect to about 45° from vertical; peduncles holding inflorescences above and beyond the foliar plane.
Texture: Smooth, glabrous.
Color: Close to 144A tinted with close to N77A.

### Reproductive Organs:

Androecium: Present on disc florets only.

Quantity per disc floret: One.
Filament length: About 1.2 mm.
Filament color: 5 Close to 145C.
Anther shape: Lanceolate.
Anther length: About 1.2 mm.
Anther color: Close to 200A.
Pollen amount: Abundant.
Pollen color: Close to 21A.
Gynoecium: Present on ray and disc florets.
Pistil length: About 5.5 mm.
Style length: About 3 mm.
Style color: Close to 153D.
Stigma shape: Bi-parted.
Stigma color: Close to 15A.
Ovary color: Close to 145D.
Seeds and fruits: Seed and fruit development have not been observed on plants of the new Bidens.

### Disease & Pest Resistance:

Plants of the new Bidens have not been shown to be resistant to pathogens and pests common to Bidens plants.

### Garden Performance:

Plants of the new Bidens have exhibited good tolerance to rain and wind and have been observed to tolerate temperatures from about 1° C. to about 47° C.

### Example 3

### Botanical Description of ‘Florbikanre’

Plants of the new Bidens have not been observed under all possible environmental conditions and cultural practices. The phenotype may vary somewhat with variations in environmental conditions such as temperature and light intensity, without, however, any variance in genotype. The following traits have been repeatedly observed and are determined to be the unique characteristics of ‘Florbikanre’. These characteristics in combination distinguish ‘Florbikanre’ as a new and distinct Bidens plant:

1. Large, upright, outwardly spreading to somewhat trailing and mounding plant habit.
2. Vigorous growth habit.
3. Freely branching habit.
4. Freely flowering habit.
5. Long flowering period.
6. Inflorescences with light red-colored ray florets and yellow-colored disc florets.
7. Strong peduncles that hold the inflorescences above and beyond the foliar plane.

Plants of the new Bidens differ primarily from plants of the parent, ‘KR-SAIKA001’ in ray floret color as inflorescences of the new Bidens have light red-colored ray florets whereas inflorescences of ‘KRSAIKA001’ have subdued red colored ray florets.

Plants of the new Bidens can be compared to plants of Bidens triplinervia ‘Florbikano’, disclosed in U.S. Plant patent application Ser. No. 13/507,488. In side-by-side comparisons conducted in Okayama-ken, Japan, plants of the new Bidens differed from plants of ‘Florbikano’ in the following characteristics:

1. Plants of the new Bidens were taller than plants of ‘Florbikano’.
2. Plants of the new Bidens had longer and thicker lateral branches than plants of ‘Florbikano’.
3. Plants of the new Bidens had longer leaves than plants of Florbikano
4. Plants of the new Bidens and ‘Florbikano’ differed in ray floret color as plants of ‘Florbikano’ had yellow orange and dark orange bi-colored ray florets.
5. Plants of the new Bidens had longer peduncles than plants of ‘Florbikano’.

### DETAILED BOTANICAL DESCRIPTION

The aforementioned photographs and following observations and measurements describe plants grown in 40-cm containers during the late spring in a polyethylene covered greenhouse in Okayama-ken, Japan and under cultural practices which approximate those generally used in commercial Bidens production. During the production of the plants, day temperatures ranged from 20° C. to 30° C. and night temperatures ranged from 5° C. to 10° C. Plants were one year old when the photographs and description were taken. In the following description, color references are made to The Royal Horticultural Society Colour Chart, 2007 Edition, except where general terms of ordinary dictionary significance are used.

### Botanical Classification:

Bidens triplinervia ‘Florbikanre’.

### Parentage:

Self-pollination of Bidens triplinervia ‘KRSAIKA001’, not patented.

### Propagation:

Type: By vegetative cuttings.

Time to initiate roots, summer: About two weeks at temperatures about 20° C. to 30° C.
Time to initiate roots, winter: About three weeks at temperatures about 10° C. to 15° C.
Time to produce a rooted young plant, summer: About three weeks at temperatures about 20° C. to 30° C.
Time to produce a rooted young plant, winter: About four weeks at temperatures about 10° C. to 15° C.
Root description: Fine, fibrous; white in color.
Rooting habit: Freely branching; dense.

### Plant Description:

Plant and growth habit: Large, upright, outwardly spreading to somewhat trailing and mounding plant habit; vigorous growth habit.

Branching habit: Freely branching habit with lateral branches potentially forming at every node.
Plant height: About 40 cm.
Plant diameter or spread: About 100 cm.

### Lateral Branches:

### Length: About 21 cm.

### Diameter: About 1.3 mm.

Internode length: About 11 cm.

Strength: Strong, flexible.
Texture: Slightly pubescent.
Color: Close to 144B tinted with close to N77A.

### Foliage Description:

Arrangement: Opposite, simple.

### Length: About 4 cm.

### Width: About 2.3 cm.

Shape: Roughly deltoid; 5 pinnatisect.

### Apex: Acute.

### Base: Acute.

Margin: Deeply incised; laciniate.

Texture, upper and lower surfaces: Smooth, glabrous.
Venation pattern: Pinnate.

### Color:

Developing leaves, upper surface: Close to 137B.

Developing leaves, lower surface: Close to 138B.
Fully expanded leaves, upper surface: Close to N137A; venation, close to 137B.
Fully expanded leaves, lower surface: Close to 137C; venation, close to 137A.

### Petioles:

### Length: About 1.2 cm.

### Diameter: About 0.6 mm.

Texture, upper surface: Sparsely pubescent.

Texture, lower surface: 5 Smooth, glabrous.
Color, upper surface: Close to N77A.
Color, lower surface: Close to 146A.

### Inflorescence Description:

Appearance: Single (daisy) inflorescence form with ray and disc florets; inflorescences positioned above and beyond the foliar plane on strong peduncles; inflorescences face mostly upright to outwardly.

Flowering habit: Freely flowering habit with numerous inflorescences developing per plant.
Fragrance: None detected.
Natural flowering season: Long flowering period, plants flower continuously from spring until the autumn in Japan.
Inflorescence longevity: Inflorescences last about one week on the plant; inflorescences persistent.

### Inflorescence Buds:

### Height: About 5 mm.

### Diameter: 5 About 4 mm.

### Shape: Ovoid.

### Color: Close to 151A.

### Inflorescence Size:

### Diameter: About 3.6 cm.

Depth (height): About 9 mm.

Disc diameter: About 4 mm.
Receptacle diameter: About 1 cm.
Receptacle height: About 3 mm.
Receptacle color: Close to 137B.

### Ray Florets:

### Length: About 1.8 cm.

### Width: About 1.1 cm.

### Shape: Ovate.

### Apex: Emarginate.

### Base: Obtuse.

### Margin: Entire.

Texture, upper and lower surfaces: 5 Smooth, glabrous.

Number of ray florets per inflorescence: Five arranged in a single whorl.
Color: When opening, upper surface: Close to 42A.
When opening, lower surface: Close to 163A and 169A.
Fully opened, upper surface: Close to 42A; towards the base, close to 28A; with development, color becomes closer to 169A and towards the base, close 15 to 163A.
Fully opened, lower surface: Close to 163B and N163A; color does not change with development.

### Disc Florets:

Shape: Tubular; apex dentate.

### Length: About 5 mm.

### Diameter: About 1 mm.

Number of disc florets per inflorescence: 5 About 42.

Color, when opening: Towards the apex, close to N167C; towards the base, close to 145B.
Color, fully opened: Towards the apex, close to 172A; towards the base, close to 145A.

### Phyllaries:

Quantity per inflorescence: Seven to eight in a single Whorl.

### Length: About 5 mm.

### Width: About 0.8 mm.

### Shape: Lanceolate.

### Apex: Acute.

### Base: Truncate.

### Margin: Entire.

Texture, upper and loWer surfaces: Smooth, glabrous.

Color, upper and loWer surfaces: Close to 137B.

### Peduncles:

Length, terminal peduncle: 5 About 19 cm.

### Diameter: About 0.9 mm.

Strength: Strong; flexible.

Aspect: Erect to about 450 from vertical; peduncles holding inflorescences above and beyond the foliar plane.
Texture: Smooth, glabrous.
Color: Close to 144B tinted with close to N77A.

### Reproductive Organs:

Androecium: Present on disc florets only.

Quantity per disc floret: One.
Filament length: About 2 mm.
Filament color: Close to 145D.
Anther shape: Lanceolate.
Anther length: About 1.6 mm.
Anther color: Close to 200A.
Pollen amount: Abundant.
Pollen color: Close to 21A.
Gynoecium: Present on ray 5 and disc florets.
Pistil length: About 6.5 mm.
Style length: About 4 mm.
Style color: Close to 153D.
Stigma shape: Bi-parted.
Stigma color: Close to 15A.
Ovary color: Close to 145D.
Seeds and fruits: Seed and fruit development have not been observed on plants of the new Bidens.

### Disease & Pest Resistance:

Plants of the new Bidens have not been shown to be resistant to pathogens and pests common to Bidens plants.

### Garden Performance:

Plants of the new Bidens have exhibited good tolerance to rain and wind and have been observed to tolerate temperatures from about 1° C. to about 47° C.

### Example 4

### Stable and Predictable Introgression of Coloration

The instant Bidens coloration and color patterns can be stably and predictably introgressed into diverse Bidens genetic backgrounds. The instant Bidens can be used as a male or female parent in crosses for introducing the trait into new Bidens plants, thereby creating diverse Bidens genetic backgrounds.

Additionally, and as known in the art, Bidens plants can be reproduced asexually by vegetative propagation or other clonal method known in the art. For example, and in no way limiting, a Bidens plant having at least one inflorescence with red ray florets, can be reproduced by (a) obtaining a tissue cutting from said plant, (b) culturing said tissue cutting under conditions sufficient to produce a plantlet with roots and shoots; and (c) growing said plantlet to produce a plant.

### Example 5

Referring to FIGS. 4 to 7, a Bidens plant according to example 5 will be described. The Bidens plant belongs to Bidens triplinervia or any progeny of Bidens triplinervia. The Bidens plant coloration and color patterns is stably and predictably introgressed into diverse Bidens genetic background, not to mention, this is a well-known conventional technique in plant fields.

Further, the Bidens plant includes any Bidens hybrid which is developed by crossing Bidens triplinervia with another Bidens type. For example, a new Bidens triplinervia, which comprises characteristics of the Bidens plant according to example 5, is created by making use of Bidens triplinervia. Then any Bidens hybrid, which is developed from the new Bidens triplinervia, is created by crossing the new Bidens triplinervia with any Bidens such as Bidens ferulifolia that is capable of crossing with Bidens triplinervia. And, a new Bidens plant such as Bidens ferulifolia, which comprises the characteristics of example 5, is created in the end. It is needless to say that producing hybrid by crossing a plant with another plant type and then developing the hybrid with unique characteristics is a well-known conventional technique in plant fields.

As shown in FIG. 4, a Bidens plant comprises ray florets and disc florets, each ray floret having an upper surface divided into a plurality of regions; wherein a color of each region is controlled based on a region-by-region approach that crosses a Bidens plant comprising ray floret coloration and patterns according to the plurality of regions with a Bidens plant comprising or not comprising ray floret coloration and patterns according to the plurality of regions. The region-by-region approach is equivalent to the section-by-section approach.

More specifically, as shown in FIG. 4, the plurality of regions includes first regions (refer to 1 in FIG. 4), a second region (refer to 2 in FIG. 4), a third region (refer to 3 in FIG. 4), and fourth regions (refer to 4 in FIG. 4), the second region being interposed between the first regions, the third region being interposed between the fourth regions, and the third region and the fourth regions being adjacent to the first regions and the second region along an axis.

Hereinafter, a method for producing the new Bidens plant according to example 5 will be described. First, the new Bidens plant with the characteristics of example 5 (that is, the ray florets is divided into four regions, the color of each region is designed by controlling flower petal color based on a region-by-region approach) is created by utilizing crossing technique and the phenomenon of mutation among Bidens plants having unique ray floret coloration and patterns according to the plurality of regions. Second, by making use of selection technology, the Bidens individuals having acquired unique characteristics are selected.

Further, a method for producing the Bidens plant according to example 5, comprises (a) crossing a first Bidens plant comprising characteristics of example 5, with a second Bidens plant that comprises or does not comprise the characteristics of example 5, and (b) selecting progeny having the characteristics. Furthermore, tissue from the Bidens plant of example 5 can be asexually propagated to produce the Bidens plant comprising the characteristics of example 5, not to mention, this asexually reproduce includes a conventional cutting from the Bidens plants. And it is needless to say that the Bidens plants can be produced from seeds having the characteristics of example 5. The seeds are sexually reproduced by utilizing the Bidens plants.

Next, the Bidens plant created by the region-by-region approach will be explained by referring to FIG. 4 to FIG. 7. As shown in FIG. 4, the color of each region is selected from a group consisting of red, yellow, orange, white, and lemon, and the color of at least one of the plurality of regions is red or orange. Further, the color of each region is selected from a group consisting of red, yellow, orange, white, lemon, black, and green, and the color of at least one of the plurality of regions is red, orange, or black.

In some cases, as shown in FIG. 4, at least one of the plurality of regions is brushed or brushed with another color.

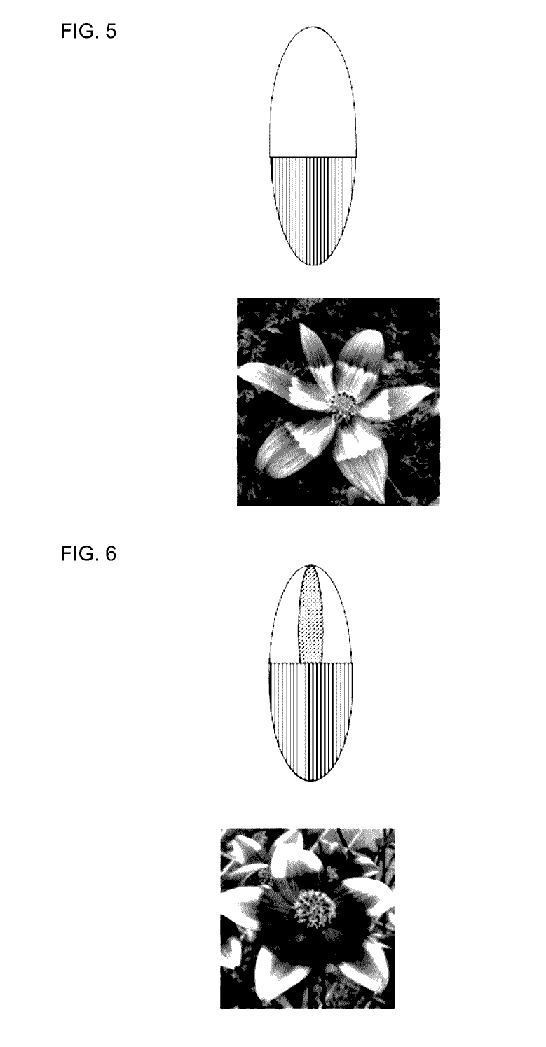

Next, the Bidens plant according to example 5 will be explained on the basis of pictures. FIG. 5 shows the Bidens plant that the color of the first regions and the second region is the same, the color of the third region and the fourth regions is the same, and the third region and the fourth regions are brushed by another color. Specifically, the color of the first regions and the second region is orange, the color of the third region and the fourth regions is orange, and the third region and the fourth regions are brushed by yellow. In this case, the ray florets is bi-color.

Further, FIG. 6 shows the Bidens plant that the color of the third region and the fourth regions is the same, the color of the first regions and the second region is the same, the color of the first regions and the second region is different from the color of the third region and the fourth regions, and the second region is brushed by the color of the third region and the fourth regions. Specifically, the color of the third region and the fourth regions is yellow, the color of the first regions and the second region is red, and the second region is brushed by red.

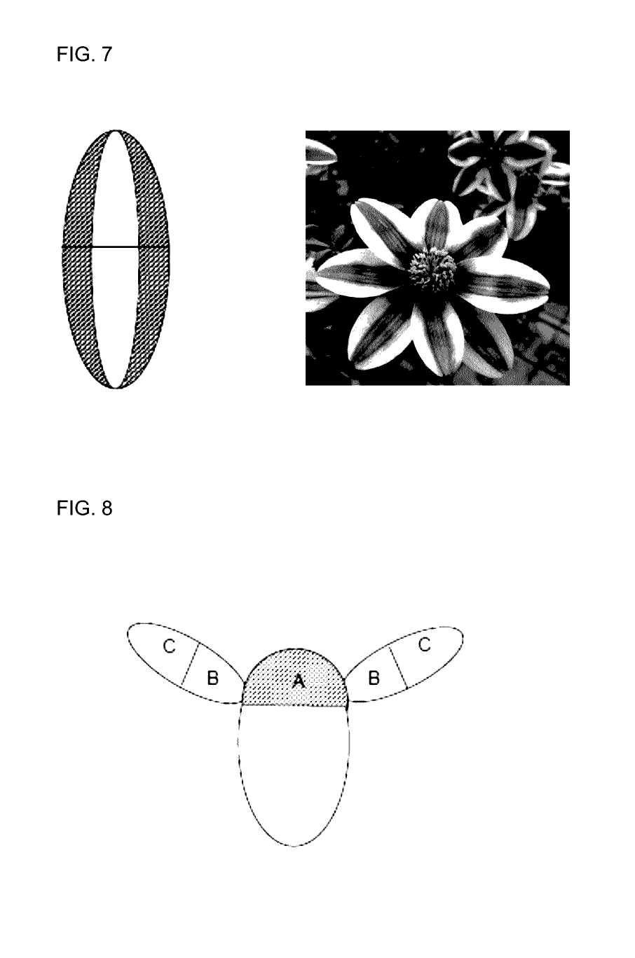

In addition, the Bidens plant according to example 5 will be explained. FIG. 7 shows the Bidens plant that the color of the first regions and the fourth regions is the same and the color of the second region and the third region is different from the color of the first regions and the fourth regions. Specifically, the color of the first regions and the fourth regions are yellow and the color of the second region and the third region are red.

### Example 6

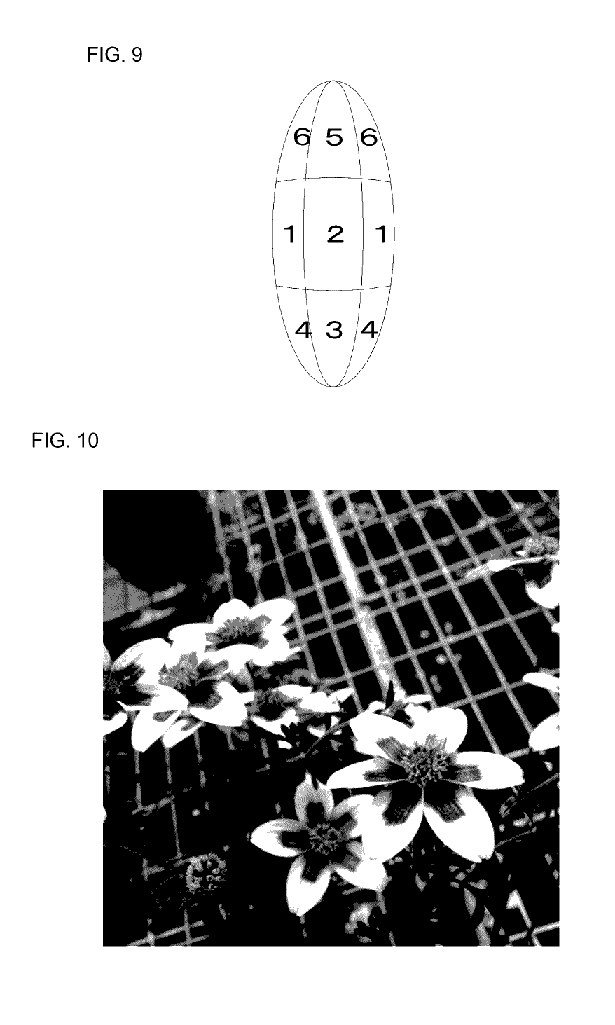

Referring to FIGS. 9 to 13, a Bidens plant according to example 6 will be described. The Bidens plant belongs to Bidens triplinervia or any progeny of Bidens triplinervia just like example 5.

As shown in FIG. 9, the plurality of regions includes first regions, a second region, a third region, fourth regions, a fifth region, and sixth regions, the second region being interposed between the first regions, the third region being interposed between the fourth regions, the fifth region being interposed between the sixth regions, and the first and the second regions being interposed between the third and the fourth regions and the fifth and the sixth regions. The Bidens plants is created by the region-by-region approach which is mentioned above. Further, a method for producing the Bidens plant according to example 6 is the same as example 5.

Moreover, the color of each region is selected from a group consisting of red, yellow, orange, white, and lemon, and the color of at least one of the plurality of regions is red or orange. Further, the color of each region selected from a group consisting of red, yellow, orange, white, lemon, black, and green, and the color of at least one of the plurality of regions is red, orange, or black. In some cases, at least one of the plurality of regions is brushed or brushed with another color.

Next, the Bidens plant according to example 6 will be explained on the basis of pictures. FIG. 10 shows the Bidens plant that the color of the first regions, the second region, and the fourth regions is yellow, the color of the third region is red, and the color of the fifth region and sixth regions is lemon.

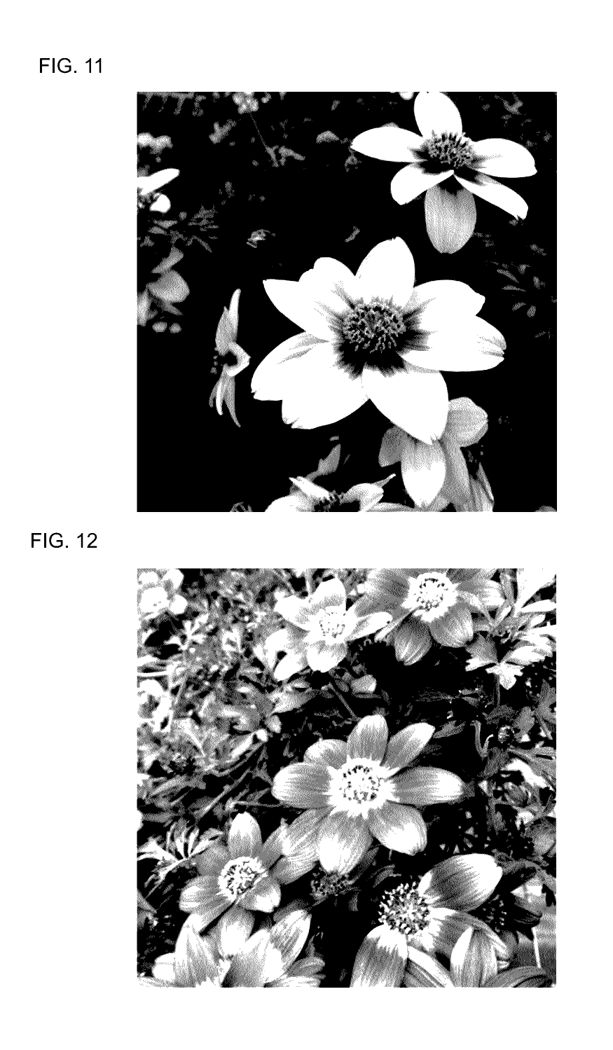

Further, FIG. 11 shows the Bidens plant that the color of the first regions and the second region is yellow, the color of the third region and the fourth regions is red, the color of the fifth region and the sixth regions is lemon.

Furthermore, FIG. 12 shows the Bidens plant that the color of the first regions, the second region, the fifth region, and the sixth regions is red and the color of the third region and the fourth regions is yellow.

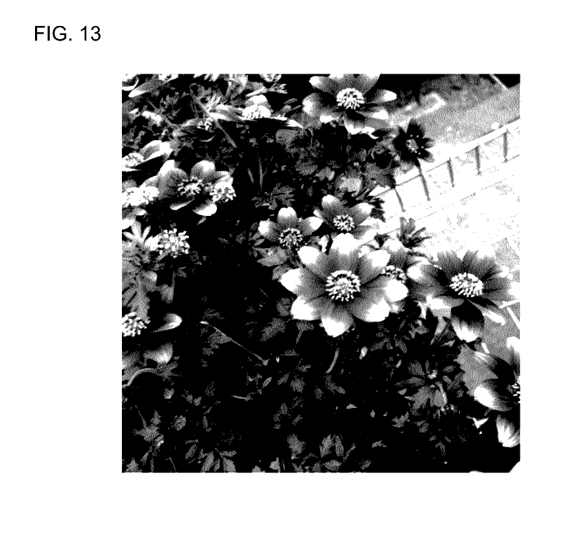

In addition, FIG. 13 shows the Bidens plant that the color of the first regions, the second region, the third region, and the fourth regions is red and the color of the fifth region and the sixth regions is lemon or lemon with red brushing.

While the foregoing has described what are considered to be the best mode and/or other examples, it is understood that various modifications may be made therein and that subject matter disclosed herein may be implemented in various forms and examples, and that they may be applied in numerous applications, only some of which have been described herein. It is intended by the following claims to claim any and all modifications and variations that fall within the true scope of the present teachings.

What is claimed is:

1. A Bidens plant comprising:
ray florets and disc florets, each ray floret having an upper surface divided into a plurality of regions;
wherein the Bidens plant belongs to Bidens triplinervia or any progeny of Bidens triplinervia; and
wherein a color of each region is controlled based on a region-by-region approach that crosses a Bidens plant comprising ray floret coloration and patterns according to the plurality of regions with a Bidens plant comprising or not comprising ray floret coloration and patterns according to the plurality of regions.

2. The Bidens plant of claim 1, wherein the plurality of regions includes first regions, a second region, a third region, and fourth regions, the second region being interposed between the first regions, the third region being interposed between the fourth regions, and the third region and the fourth regions being adjacent to the first regions and the second region along an axis.

3. The Bidens plant of claim 2, wherein the color of each region is selected from a group consisting of red, yellow, orange, white, and lemon, and the color of at least one of the plurality of regions is red or orange.

4. The Bidens plant of claim 2, wherein the color of each region is selected from a group consisting of red, yellow, orange, white, lemon, black, and green, and the color of at least one of the plurality of regions is red, orange, or black.

5. The Bidens plant of claim 3, wherein at least one of the plurality of regions is brushed or brushed with another color.

6. The Bidens plant of claim 5, wherein the color of the first regions and the second region is the same, the color of the third region and the fourth regions is the same, and the third region and the fourth regions are brushed by another color.

7. The Bidens plant of claim 5, wherein the color of the third region and the fourth regions is the same, the color of the first regions and the second region is the same, the color of the first regions and the second region is different from the color of the third region and the fourth regions, and the second region is brushed by the color of the third region and the fourth regions.

8. The Bidens plant of claim 3, wherein the color of the first regions and the fourth regions is the same and the color of the second region and the third region is different from the color of the first regions and the fourth regions.

9. The Bidens plant of claim 2, the color of at least one of the plurality of regions is gradually changing from beginning to bloom towards ending to bloom.

10. The Bidens plant of claim 1, wherein the plurality of regions includes first regions, a second region, a third region, fourth regions, a fifth region, and sixth regions, the second region being interposed between the first regions, the third region being interposed between the fourth regions, the fifth region being interposed between the sixth regions, and the first and the second regions being interposed between the third and the fourth regions and the fifth and the sixth regions.

11. The Bidens plant of claim 10, wherein the color of each region is selected from a group consisting of red, yellow, orange, white, and lemon, and the color of at least one of the plurality of regions is red or orange.

12. The Bidens plant of claim 11, wherein the color of each region is selected from a group consisting of red, yellow, orange, white, lemon, black, and green, and the color of at least one of the plurality of regions is red, orange, or black.

13. The Bidens plant of claim 11, wherein at least one of the plurality of regions is brushed or brushed with another color.

14. The Bidens plant of claim 10, wherein the color of the first regions, the second region, and the fourth regions is yellow, the color of the third region is red, and the color of the fifth region and sixth regions is lemon.

15. The Bidens plant of claim 10, wherein the color of the first regions and the second region is yellow, the color of the third region and the fourth regions is red, the color of the fifth region and the sixth regions is lemon.

16. The Bidens plant of claim 10, wherein the color of the first regions, the second region, the fifth region, and the sixth regions is red and the color of the third region and the fourth regions is yellow.

17. The Bidens plant of claim 10, wherein the color of the first regions, the second region, the third region, and the fourth regions is red and the color of the fifth region and the sixth regions is lemon or lemon with red brushing.

18. The Bidens plant of claim 1, wherein the Bidens plant coloration and color patterns are stably and predictably introgressed into diverse Bidens genetic background.

19. A method for producing the Bidens plant of claim 1, comprising:
(a) crossing a first Bidens plant comprising the characteristics of claim 1, with a second Bidens plant that comprises or does not comprise the characteristics of claim 1, and
(b) selecting progeny having the characteristics of claim 1.

20. A method for asexually reproducing the Bidens plant of claim 1, comprising:
(a) obtaining a tissue cutting from said plant,
(b) culturing said tissue cutting under conditions sufficient to produce a plantlet with roots and shoots; and
(c) growing said plantlet to produce a plant.
